(19) United States
(12) Patent Application Publication
(10) Pub. No.: US 2006/0258021 A1
(43) Pub. Date: Nov. 16, 2006

(54) Biocompatible linkers for surface plasmon resonance biosensors

(75) Inventors: Karl S. Booksh, Phoenix, AZ (US); Jean-Francois Masson, Decatur, GA (US)

Correspondence Address: QUARLES & BRADY LLP, RENAISSANCE ONE, TWO NORTH CENTRAL AVENUE, PHOENIX, AZ, 85004-2391, US

(73) Assignee: Arizona Board of Regents, a body corporate, acting for and on behalf of Arizona State University, Tempe, AZ (US)

(21) Appl. No.: 10/567,059

(22) PCT Filed: Aug. 12, 2004
(86) PCT No.: PCT/US04/26437

§ 371 Date: Feb. 1, 2006

Related U.S. Application Data
(60) Provisional application No. 60/494,389, filed on Aug. 12, 2003.

(51) Int. Cl. G01N 1/28; G01N 33/543

(52) U.S. Cl. 436/518; 427/2.11

(57) ABSTRACT

A method of coating an SPR biosensor specific for an analyte to reduce protein fouling, the method has the steps of providing an SPR biosensor, providing a solution of 11-mercaptoundecanol; incubating the SPR biosensor in the 11-mercaptoundecanol solution to form a self-assembling monolayer (SAM); incubating the SPR with SAM in a solution of epichlorohydrin and diglyme; next incubating the SPR in ethanolamine; preparing a solution of EDCNHS and a biocompatible polymer; incubating the SPR from ethanolamine in the EDC/NHS/polymer solution; providing a ligand specific for the analyte in a solution; incubating the polymer-coated SPR in the ligand solution to permit the ligand to react with the polymer-coated SPR; washing the ligand-coated SPR to remove unreacted ligand, thereby providing an SPR capable of reacting with the analyte. Another method replaces the solution for the SAM layer with a solution of MHA or NHS-MHA with HT, and attaches the ligand to the resulting SAM layer.

### TECHNICAL FIELD

The present invention is in the field of medical diagnostics and more particularly relates to biocompatible materials suitable for avoiding bodily fluid and tissue interaction with an implanted diagnostic device.

### BACKGROUND

The use of polymeric supports for SPR sensors has been restricted mainly to CM-dextran, although some studies have used streptavidin, polylysine, polyethyleneglycol (PEG), and polyvinylphenylboronic acid as a support layer. Most recent SPR studies include the binding and adsorption interactions of polymers, the optical properties of polymers, the growth monitoring of polymers, the hydration properties, and the use of molecularly imprinted polymers as molecular recognition elements. Recent work demonstrated the use of chitosan, dextran, poly(oxyethylene), poly(ethyleneimine), and poly(acrylamide) as well as a high density PEG for immunoprobes on a glass slide using contact angle measurements. Another investigated the use of carboxylated poly(vinyl chloride), polystyrene, and chloropropyl-modified sol-gel for a direct evanescent wave immunoassay using total internal reflectance fluorescence. However, these polymers were only coated on a fiber optic instead of covalently attached to the surface. Polymer layers are subject to non-specific binding.

SPR theory has been extensively described. Light undergoing total internal reflection exhibits an evanescent wave. This evanescent wave can excite a standing charge on a thin gold film (FIG. 1). The gold film is typically 50 nm thick. In order for the standing charge excitation on the gold film to occur, it must be in contact with a sample of lower refractive index than the wave guide. In order for this to occur, the wave vector of the standing charge ksp and the wave vector of the evanescent wave k_{X} must be equal (Equations 1a and 1b).

$$\begin{matrix}{k}_{sp} = {k}_{o} \sqrt{\frac{{ɛ}_{m} {ɛ}_{S}}{{ɛ}_{m} + {ɛ}_{S}}} & ( 1 a ) \\ {k}_{x} = {k}_{o} {η}_{D} sin {Θ}_{inc} & ( 1 b )\end{matrix}$$

Where kₒ is the wave vector of the incident light, εₘ and εₛ are the complex dielectric constants of the metal and the sample respectively, η_{D} is the refractive index of the wave guide and Θ_{inc} is the incident angle of the light.

Multiple combinations of incident light angles and wavelengths can excite the standing charge. When this occurs, the photon is absorbed, shown by a minimum in the reflection spectra (FIG. 2). The position of the minimum (λ_{SPR}) is indicative of the dielectric constant or the refractive index within 100-200 nm of the gold film. SPR is most sensitive for processes occurring at the surface. The sensitivity decreases exponentially for processes occurring further from the surface.

The major challenge to overcome before the use of SPR in complex solutions is to reduce or eliminate sensor fouling. SPR measures any change of refractive index at the probe surface, so non-specific binding will produce an undistinguishable signal from specific binding. In the case of SPR-based immunoassays, proteins and cells will create an overwhelming signal, 10-100 times more intense than the signal from the antigen. CM-dextran has failed as a support when antigens are to be detected in bovine serum due to its inability to control non-specific binding. Biocompatible polymers have been used to reduce cell and protein fouling on implantable devices. Even when cells were filtered out using a mesh around the probe, protein fouling was still present.

There is clearly a need for ligand supports that will enhance the SPR signal by increasing the number of adsorption sites and minimizing non-specific binding, allowing SPR sensors to be used in complex fluids like serum, blood or wounds.

### SUMMARY OF INVENTION

In one embodiment, there is provided a method of coating an SPR biosensor specific for an analyte to reduce protein fouling. The method includes providing an SPR biosensor, providing a solution of 11-mercaptoundecanol; incubating the SPR biosensor in the 11-mercaptoundecanol solution to form a self-assembling monolayer (SAM); incubating the SPR with SAM in a solution of epichlorohydrin and diglyme; next incubating the SPR in ethanolamine; preparing a solution of EDC/NHS and a biocompatible polymer; incubating the SPR from ethanolamine in the EDC/NHS/polymer solution; providing a ligand specific for the analyte in a solution; incubating the polymer-coated SPR in the ligand solution to permit the ligand to react with the polymer-coated SPR; and washing the ligand-coated SPR to remove unreacted ligand, thereby providing an SPR capable of reacting with the analyte.

In another embodiment, the biocompatible polymer is prepared from carboxymethylated hyaluronic acid, OPSS-PEG-NHS, alginic acid, humic acid, polymethacrylate co-vinyl acetate or polyacrylic co-vinyl acetate. The analyte can be an antigen and the ligand can be an antibody. In another embodiment, the antigen is cardiac myoglobin and the antibody is anti-myoglobin. In yet another embodiment, the antigen is cardiac troponin I and the antibody is anti-cardiac troponin I. In yet another embodiment, the antigen is interleukin-6 (IL-6) and the antibody is anti-IL-6, whereby the biosensor can monitor wound healing. In yet another embodiment, the antigen is NSE and the antibody is anti-NSE, whereby the biosensor can monitor patients for ischemic stroke. In yet another embodiment, the antigen is S-100B and the antibody is anti-S-100B, whereby the biosensor can monitor patients for ischemic stroke. In yet another embodiment, the antigen is SMN1-4 and the antibody is anti-SMN1-4, and further comprising the step of preparing a cellular extract, whereby a low value is indicative of spinal motor atrophy.

In another embodiment, there is provided a method of coating an SPR biosensor specific for an analyte to reduce protein fouling. The method has the steps of providing an SPR biosensor; providing a solution of MHA or NHS-MHA with HT; incubating the SPR biosensor in the MHA-HT solution for a time sufficient to permit the formation of SAM; providing a solution of a ligand specific for the analyte; incubating the SPR biosensor with SAM in the ligand solution for a time sufficient for the ligand to react with the SAM, thereby providing the biosensor with ligands specific for the analyte. The analyte may be an antigen and the ligand an antibody. The antigen can be cardiac myoglobin and the antibody can be anti-myoglobin. Alternatively, the antigen can be cardiac troponin I and the antibody can be anti-cardiac troponin I. To monitor wound healing, the antigen can be interleukin-6 (IL-6) and the antibody is anti-IL-6. In yet another embodiment, to monitor patients for ischemic stroke, the antigen can be NSE and the antibody is anti-NSE; or the antigen is S-100B and the antibody is anti-S-100B. In yet another embodiment, the antigen is SMN1-4 and the antibody is anti-SMN1-4; and a further step comprising preparing a cellular extract. In this last embodiment, a low value is indicative of spinal motor atrophy.

Finally, additional features of the disclosed methods are described in detail below and in the appended claims.

### BRIEF DESCRIPTION OF THE DRAWINGS

FIG. 1 illustrates SPR theory. It shows light excitation of a standing charge on a thin metal film.

FIG. 2 is a graph showing an SPR signal at constant angle.

FIG. 3 illustrates an optical fiber SPR sensor in size comparison with a dime.

FIGS. 4A and 4B are sensorgrams for the polymer attachment to the SPR sensor. FIG. 4A shows the results for humic acid attachment to the free amine and FIG. 4B shows the results for the polymethacrylic acid-co-vinyl acetate polymer.

FIGS. 5A through 5I show the GAIR-FTIR spectra for OPSS-PEG-NHS (5A), alginic acid (5B), CM-dextran (5C), CM-hyaluronic acid (5D), Hyaluronic acid (5E), PMAVA (5F), humic acid (5G), polylactic acid (5H) and thiol-amine linker (5I).

FIG. 6 is an example of a sensorgram for the antibody binding to the polymers.

FIG. 7 shows an antigen binding curve.

FIG. 8 is a graph of the antibody shift v. the MG shift, illustrating the correlation between the two parameters.

FIG. 9 is a graph showing the shift caused by exposure to serum over time for CM-dextran.

FIG. 10 graphs the log of the Dextran molecular weight against the shift due to serum fouling of an SPR sensor with CM-dextran (gray) and anti-MG on the surface (black).

FIG. 11 is a bar graph comparing CM-dextran 500 with alginic acid, CM-hyaluronic acid and hyaluronic acid.

FIGS. 12A and 12B show that the signal when the sensor is placed in contact with bovine serum (84 mg/ml protein) at 25° C. (12A) is much larger than the signal for cTnI detection at 10 ng/ml (12B). The signal from serum protein adsorption (FIG. 12A) did not reach equilibrium after 10 minute exposure, but adsorption is partially reversible in HBS. The signal for cTnI binding (FIG. 12B) reached equilibrium after less than five minutes.

FIG. 13 illustrates the kinetics for CM-dextran 500 non-specific binding (NSB) of serum protein for 14 days at 0° C. The shift is referenced to the first data point acquired when the sensor was exposed to bovine serum. The shift reported in Table 3 is an average of the shift for the final five days of the experiment.

FIG. 14 is a graph showing layer formation shift for MHA mixed with HT (black) and NHS-MHA mixed with HT (gray). Percent MHA and % NHS-MHA represent the solution composition placed in contact with the probes. The actual layer composition differs from the solution.

FIG. 15A and 15B show the GATR-FTIR spectra of self-assembled matrices prepared from 100% MHA (FIG. 15A) and 100% NHS-MHA (FIG. 15B) layers on a gold slide. The C═O region shows major differences between 100% MHA and 100% NHS-MHA confining the presence of the NHS group on the surface.

FIG. 16 graphs the shift due to NSB and percent NSB compared to CM-dextran 500 versus the % MHA or NHS-MHA. NSB of bovine serum for NHS-MHA sensors (black squares), MHA sensors using the cold antibody (light gray circles), and MHA sensors using the hot antibody attachment (gray triangles). Less NSB was observed for MHA at higher MHA surface coverage for both the hot and cold antibody attachment. NSB did not differ for NHS-MHA at low or high surface coverage.

FIG. 17 shows SPR sensor performance in detecting a 25 ng/mL MG solution in HBS pH 7.4 for different MHA layer compositions. A two-fold increase in the sensor response using the cold (black squares) antibody attachment was noted compared to the hot (gray) antibody attachment. The cold and hot antibody attachment results were statistically different.

FIG. 18 shows SPR sensor sensitivity for MHA (gray squares) and NHS-MHA (black squares) using the cold antibody reaction. Percent MHA and % NHS-MHA refer to the solution composition during sensor preparation. A concentration dependent profile for NHS-MHA can be observed and correlated to the surface coverage of NHS-MHA. MHA does not have any surface coverage dependent profile.

FIG. 19 graphs a measurement cycle starting in HBS pH 7.4, moving to an antigen solution (cTnI; 5 ng/mL) and returning to HBS. The sensor quickly regenerated.

FIG. 20 graphs a Langrnuir isotherm for alginic acid (diamonds), CM-dextran (circles), 100% NHS-MHA (triangles) and OPSS-PEG-NHS (squares). The calibration range covers the biologically relevant range of myoglobin (MG); (15-30 ng/mL) during myocardial infarctions.

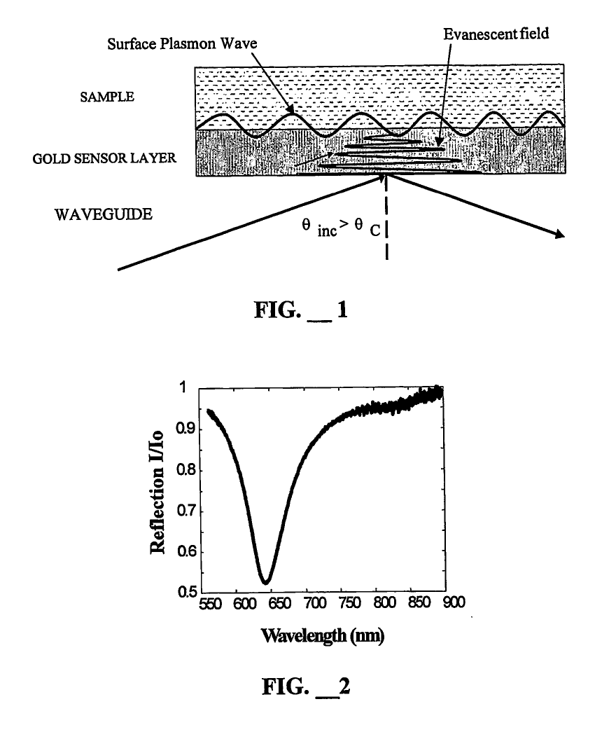

### DETAILED DESCRIPTION OF THE PREFERRED EMBODIMENTS

This invention concerns the preparation and use of SPR sensors with different biocompatible polymers to eliminate non-specific fouling. The biocompatible polymers must have carboxylic acids on their backbone to allow antibody attachment and must be able to attach a sufficient amount of antibodies to allow the antigen detection at biologically relevant concentrations. Humic acid, hyaluronic acid, carboxymethylated hyaluronic acid (CM-hyaluronic acid), alginic acid, polyacrylic acid, OPSS-PEG-NHS and PMAVA are biocompatible and can attach antibodies. dl-polylactic acid (PLA) is a biocompatible polymer without any carboxylic acids on the backbone. Since it cannot bind antibodies but can have an impact on non-specific binding to SPR probes, PLA is used as a reference.

Herein, the sensor performance for quantitative detection of analytes (e.g., myoglobin (MG)) using different biopolymers and different self assembled layers is explored. MG is an important marker in myocardial infarctions (MIs), which are a leading cause of death in the United States. During a myocardial infarction, the cardiac muscles are damaged; and proteins or cardiac markers are released from these muscles. Currently, multiple blood samples are collected at different time intervals; and cardiac marker levels are monitored in vitro to detect MI. This approach requires several hours to provide a definitive diagnosis of infarction A sensor that can monitor the cardiac markers myoglobin (MG) and cardiac Troponin I (cTnI) in less than 10 minutes would improve patient care by allowing a quickly definitive diagnosis of MI and minimize utilization of costly medical resources. SPR has detected cardiac markers MG and cardiac troponin I (cTnI) at biological levels in HBS pH 7.4 in less than 10 minutes.

The sensor preparation is described in Example 1. The biopolymer attachment has been monitored using SPR and FTIR. Antibodies for MG have been attached to tested biopolymers, except for polylactic acid, which acts as a reference. The performances of the sensors during the detection of MG from a 25 ng/mL MG solution in HBS pH 7.4 were also compared. Fouling reduction in bovine serum was tested for the polysaccharides CM-dextran, CM-hyaluronic acid, hyaluronic acid, and alginic acid and other combinations.

The base material can include at least one of gold, stainless steel, tantalum, titanium, nitinol, platinum, iridium, silver, tungsten, or another biocompatible metal, or alloys of any of these; carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradable polymer, or mixtures or copolymers of these; a protein, an antibody or extracellular matrix component, collagen, fibrin or another biologic agent; or a suitable mixture of any of these. Gold is particularly useful as the base material for an SPR biosensor.

Also useful in some special instances are monoacrylates such as n-butyl-acrylate, n-butyl methacrylate, 2-ethylhexyl acrylate, lauryl-acrylate, and 2-hydroxy-propyl acrylate. Small quantities of amides of (meth)acrylic acid such as N-methylol methacrylamide butyl ether are also suitable, N-vinyl compounds such as N-vinyl pyrrolidone, vinyl esters of aliphatic monocarboxylic acids such as vinyl oleate, vinyl ethers of diols such as butanediol-1, 4-divinyl ether and allyl ether and allyl ester are also suitable. Also included would be other monomers such as the reaction products of di- or polyepoxides such as butanediol-1, 4-diglycidyl ether or bisphenol A diglycidyl ether with (meth)acrylic acid. The characteristics of the photopolymerizable liquid dispersing medium can be modified for the specific purpose by a suitable selection of monomers or mixtures thereof.

Other useful polymer systems include a polymer that is biocompatible and minimizes irritation in the body when the biosensor is implanted. The polymer may be either a biostable or a bioabsorbable polymer depending on the desired rate of release or the desired degree of polymer stability, but a bioabsorbable polymer can be used in this embodiment since, unlike a biostable polymer, it will not be present long after implantation to cause any adverse, chronic local response. Bio-absorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used, such as polyolefins, polyisobutylene and ethylene-alpha-olefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styiene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

### EXAMPLES

### Example 1

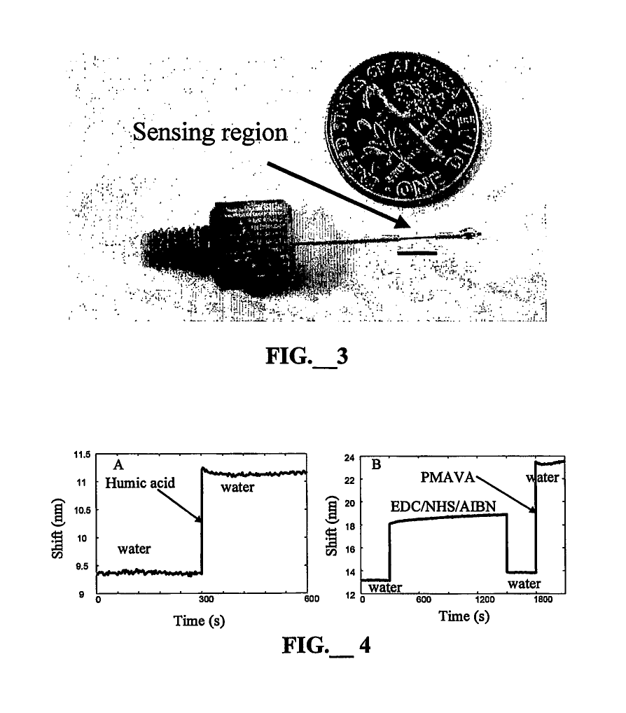

The manufacture of the SPR sensors used in this study has been described previously (L. A. Obando and K. S. Booksh, Anal. Chem., 1999, 71:5116). Here 400-micron diameter multimode fiber optics were employed for the sensor tip. However, multimode fibers as narrow as 50 microns can also be used. In the current configuration, fibers 45 mm long were cleaved. An 11-mm long piece of the buffer protecting the fiber was removed, and 5 mm is replaced to protect the mirror on the distal end (FIG. 3). The distal end was polished with 5 micron and 1 micron lapping films. The distal end was then washed with isopropanol and the sensor was dried at 100° C. for 10 minutes. A 5 nm adhesion layer of chromium (Cr) was sputtered on the distal end of the sensor, and a 50 nm layer of gold (Au) was deposited to form a mirror. The mirror was sealed by oven cured epoxy. Ten to 15 mm of the buffer on the other end of the fiber was removed. The fiber was installed on the connector and fixed in place using oven curing. The connector end was polished using 9, 5 and 1 micron lapping films. The cladding on the sensing area was removed using acetone. The sensor was visually inspected using a microscope objective to ensure that all the cladding has been removed. Five um of Cr and 50 nm of Au was deposited on the sensing area. The sensor was rotated while being sputtered to ensure an even layer of Au. The probe performance was tested in ethanol. FIG. 3 presents one of the fiber optic probe tips to scale. Two 200-micron diameter fibers were fitted into the custom design adaptor; one fiber brought light from the white LED employed as a source, while the other returned the reflected light to the spectrometer and CCD detector. A Jobin-SPEX 270 M spectrometer with an 1800 g/mm grating was used to narrow the spectral range to 42.8 um. The spectra were collected with an Andor CCD camera. A resolution of 0.0421 nm/pixel was obtained.

### Example 2

Ge Attenuated Total Reflection Fourier Transform Infra-Red Spectroscopy System

The polymer attachment on the gold surface was monitored using GATR-FTIR. The analysis of the polymer coated glass slides was performed using a Bruker IFS66v/s FTIR with an MCT detector cooled by liquid nitrogen (Billerica, Mass.). A Harrick GATR attachment (Ossining, N.Y.) was also used. The germanium crystal was washed with methyl ethyl ketone and the coated glass slides were placed face down on the crystal. The GATR attachment was placed in the FTIR and the compartment was evacuated to 1 mbar. Transmission spectra were comprised of the average of 1024 scans with the background subtracted. Precleaned glass slides were washed with acetone. A 5 nm layer of Cr and 50 nm layer of Au were deposited on the glass slide. The slides were modified chemically as described below. Upon completion of the reactions, the polymer coated gold slides were washed with ethanol and dried with compressed air. The slides were then analyzed by GATR-FTIR.

The polymers on the probes have a higher refractive index than the water into which the probes were immersed. The polymers induced a red shift when they are attached to the surface compared to the signal of a stable intermediate in water alone. The amount of red shift can be related to the surface coverage of the polymer, with larger red shifts signifiing larger amounts of polymer on the surface. The reaction conditions disclosed below were optimized to maximize this shift.

### Example 3

Preparation of Polymer layers

3.a. CM-dextran, CM-hyaluronic Acid and Hyaluronic Acid Layer Preparation

The synthesis of these layers was based on the CM-dextran chemistry used elsewhere for protein immobilization on an SPR surface (S. Lofas and B. Johnsson, J. Chem. Soc. Chem. Comm., 1990,21:1526; B. Johnsson, S. Lofas and G. Lindquist, Anal. Biochem., 1991, 198:268). All reactions occurred in aqueous solution without any stirring or shaking. The bare gold surface on the SPR probe was contacted overnight with 0.005 M 11-mercaptoundecanol in an 80:20 solution of ethanol and water to form a self-assembled monolayer (SAM). This SAM was reacted with 0.6 M epichlorohydrin in a 1:1 mixture of diglyme and 0.4 M NaOH for four hours. This layer was washed with water, ethanol and water again. The surface was reacted for 20 hours with an aqueous solution containing 0.3 g/mL dextran or 0.3 g/mL hyaluronic acid (Fisher, Hampton, N.H.) and 0.1 M NaOH. Stopping at this stage produced a hyaluronic acid layer on the sensor after hyaluronic acid treatment. The resulting matrix was modified to a carboxymethylated matrix by reaction with 1 M bromoacetic acid in 2 M NaOH for 16 hours.

3.b. Alginic Acid, Humic Acid, dl-Polylactic Acid and Polyacrylic Acid Layer Preparation

The bare gold surface on the SPR probe was contacted overnight with 0.005 M 11-mercapto-undecanol in an 80:20 solution of ethanol and water to form a self-assembled monolayer (SAM). This SAM was reacted with 0.6 M epichlorohydrin in a 1:1 mixture of diglyme and 0.4 M NaOH for four hr. This layer was washed with water, ethanol and water again. The probe was contacted with a 1 M ethanolamine solution at pH 8.5 for 20 hours. The sensor was then equilibrated for 15 min in water. Meanwhile, a 1:1 solution of 0.4 M EDC (N-ethyl-N′-(3-dimethylaminopropyl) carbodiimide hydrochloride) and 0.1 M NHS (N-hydroxysuccinimide) was reacted for 5 min. A 10 mg/mL solution (alginic acid, Aldrich, Milwaukee, Wis.; polyacrylic acid, Polysciences, Warrington, Pa.), or 5 mg/mL solution (dl-polylactic, Polysciences), or 2 mg/mL (humic acid, Aldrich, Milwaukee, Wis.) was mixed 1:1 with the EDC-NHS solution and equilibrated for 10 min. The sensor was reacted with the polymer-EDC-NHS solution at 50° C. for 16 hours for alginic acid, humic acid and polylactic acid, and 20 min at 50° C. for polyacrylic acid.

3.c. PMAVA Layer Preparation

The bare gold surface on the SPR probe was contacted overnight with 0.005 11-mercaptoundecanol in an 80:20 solution of ethanol and water to form a self-assembled monolayer (SAM). This SAM was reacted with 0.6 M epichlorohydrin in a 1:1 mixture of diglyme and 0.4 M NaOH for 4 hrs. This layer was washed with water, ethanol and water again. The probe was contacted with a 1 M ethanolamine solution at pH 8.5 for 20 hr. The sensor was then equilibrated for 15 min in water. Meanwhile, a 1:1 solution of 0.4 M EDC and 001 MNHS was reacted for 5 min. A 10 mg/mL solution of 4,4′ azobis(4-cyanovaleric acid) was mixed 1 :1 with the EDC-NHS solution and equilibrated for 10 min. 4,4′ Azobis(4-cyanovaleric acid) does not fully dissolve in water. The suspension was used as it was. Then the sensor was reacted with the 4,4′azobis(4-cyanovaleric acid)-EDC-NHS solution at toom temperature for 20 min. The sensor was washed in water for 5 min. The sensor was placed in a hot solution, 60° C., of 0.5 mL of methacrylic acid, 0.5 mL of vinyl acetate and 1 mL of ethanol. Then the temperature was increased to 80° C. and maintained until the polymerization began in the solution and the solution boiled. This process required 5 min to occur, with the phenomena occurring simultaneously. The probe was finally rinsed in ethanol.

3.d. OPSS-PEG-NHS Layer Preparation

OPSS-PEG-NHS is a custom synthesis from Nektar (Huntsville, AL). It was used as described by Hirsch et al. (J. R. Hirsch, et al., Anal Chem, 2003, 75:2377). OPSS-PEG-NHS reacted overnight at 4° C. with anti-MG in 100 mM NaHCO₃, at pH 8.5. The concentration of anti-MG and PEG-NHS were 1.2 mg/mL. Two hundred μL of the OPSS-PEG-anti-MG solution was diluted to 2 mmL with 1.8 mM K₂CO₃, and the gold probes were reacted for 24 hr at 4° C.

### Example 4

Anti-MG Attachment to the Sensor

After the polymers were immobilized on the probes, their surfaces were activated by immersion in 1:1 aqueous solutions of 0.4 M EDC and 0.01 M NHS for 10 min. An amine coupling was performed on this activated surface by reaction with a 700 μg/mL solution of human anti-myoglobin (ICN Biochemicals, polyclonal rabbit antiserum to human MG, KA and kA were not available) at pH 4 (10 mM sodium acetate buffer) and 37° C. for 20 minutes. Next, non-specifically bound proteins were washed away and the non-reacted sites on the polymers were deactivated by rinsing the probes with an aqueous solution of 1M ethanolamine at pH 8.5 for 10 minutes. Finally, the probes were dipped in 25 ng/mL buffered aqueous solutions of MG to test their performance. The measurement was done in a static solution at 25° C. The temperature was controlled to about 0.5° C. in a water bath.

### Example 5

Sensor Fouling

The technique used to measure serum fouling has been previously described. The sensors with CM-dextran, CM-hyaluronic acid, hyaluronic acid and alginic acid were prepared as described above. Anti-MG functionalized sensors were then placed in a bovine serum solution at 0° C., and measurement Of XSPR was performed daily for 14 days.

### Example 6

SPR Analysis

Table 1 summarizes the shift for the six polymers used above. The shift was used to compare the antibody binding to the sensor instead of the surface coverage because the relative performance was compared. Because polymers are three-dimensional structures, using a two-dimensional model of the antibody coverage of the polymer matrix is less meaningful than actual performance. The shifts for the polysaccharides were not monitored because the reaction did not allow an easy intermediate and stable step before the polymer attachment to the sensor. Nevertheless, the shift varied from 1.8 nm to 9.5 mn. The smallest shift was for humic acid, apparently because only a fraction of humic acid reacted with the surface. After reaction in the vial at the high reaction temperature of 50° C., a precipitate was found, which constituted a large fraction of agglomerated humic acid. OPSS-PEG-NHS has a similar molecular weight to humic acid, but the reaction sequence required the antibody reaction prior to the polymer attachment to the sensor. Therefore, the shift reported in Table 1 for OPSS-PEG-NHS included the shift induced by the antibody. FIG. 4A shows the kinetic sensorgram for a water sample before and after the reaction of humic acid with the surface. The binding reaction could not be monitored in real-time due to the absence of an in-line temperature controller other than room temperature control exerted with a water bath. Therefore, water was used as a reference point, and the shift presented in Table 1 was calculated using water as a reference point. The stability of the water signal indicated that the polymer attaching to the surface caused the shift. FIG. 4B shows the sensorgram for the preparation of the PMAVA sensor.

An on-sensor polymerization technique has been developed. The initiator was attached to the surface as shown in the EDC/NHS/AIBN part of the sensorgram. Then the sensor was immersed in the polymerization solution containing the monomers, vinyl acetate and methacrylic acid, and ethanol solvent. The polymerization occurs at high temperature by the radical breakdown of the initiator. This data is not shown. A sensor without the initiator was prepared and no difference in the SPR signal was noted as well as no polymerization. Finally, the sensor signal was measured again in the reference water solution to measure the shift. The polymerization on the probe seeds polymerization in the bulk solution. The polymer created by this process was collected and analyzed using Raman spectroscopy to confirm the reaction.

TABLE 1
SPR shift resulting from polymer attachment on the probe

| Polymer | Shift (nm) | Polymer | Shift (nm) |
|---|---|---|---|
| OPSS-PEG-NHS | 6.6* | Humic acid | 1.8 |
| Polyacrylic acid | 2.2 | Polylactic acid | 9.5 |
| PMAVA | 9.5 | | |

*Includes the shift from the antibody binding on the polymer

### Example 7

GATR-FTIR Characterization

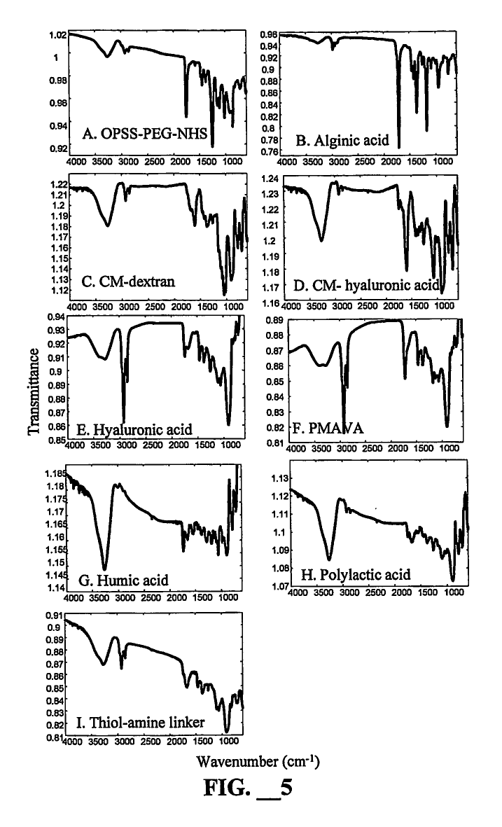

The initial GATR-FITR experiment was performed on gold-coated glass slides instead of the fiber-optic based sensors described above. FIG. 5 shows the GATR-FTIR spectra for every polymer attached to the SPR sensor. The GATR-FTIR was performed on 8 of 9 coatings. This technique was not needed to verify the attachment of polyacrylic acid polymer because the polymer was visible on the surface. Two regions of interest were monitored. Each polymer shared similar bands from the presence of carboxylic acids on its backbone. The uniqueness lay in the band position and relative intensity.

The regions of C═O vibration, around 1650 cm⁻¹ and 1750 cm⁻¹, were analyzed to assess the carboxylic acid, amide, and ester bands of the polymer. The C—H regions around 3000 cm⁻¹ were also analyzed. Finally, a comparison was done to ensure the uniqueness of the fingerprint region between 1400 cm⁻¹ and 1000 cm⁻¹. A close comparison of some similar polysaccharides (e.g., alginic acid, CM-dextran, CM-hyaluronic acid and hyaluronic acid (FIGS. 5B, C, D and E, respectively) showed distinctive differences in the regions of interest. Although they had the characteristic bands at 1700 cm⁻¹, the relative intensity was different for each polymer; and the fingerprint regions differed greatly. OPSS-PEG-NHS, PMAVA, humic acid and polylactic acid (FIGS. 5A, F, G and H, respectively) all had distinct spectra. They also differed from the thiol-amine linker used to attach the polymer to the gold surface (FIG. 51).

### Example 8

Analyte-Sensitive Properties

Two different approaches were used to monitor the sensor's performance. First, the degree of shift caused by the antibody attachment was an indication of the antibody surface coverage on the sensor. Second, the amount of shift caused by probe immersion in a 25 ng/mL saline solution of MG was used to monitor the sensor's performance.

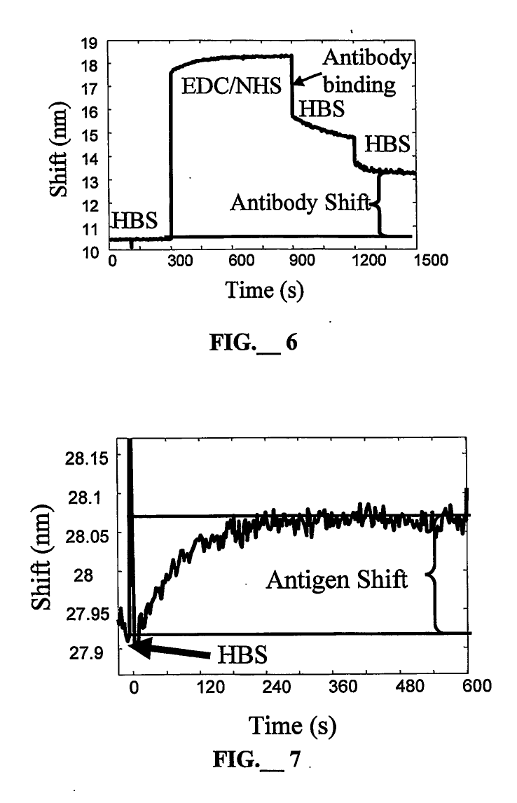

The antibody loading was performed as previously optimized (J -F. Masson, L. Obando, S. Beaudoin and K. Booksh, Talanta, 2004, 62:865), with the reaction at pH4 and 37° C. It was optimized for the CM-dextran polymer. The shift reported in Table 2 was calculated from the sensors' response in a reference media, HEPES-buffered saline pH 7.4 (HBS), before and after the reaction with the antibody. FIG. 6 shows how the shift was calculated. The shift for OPSS-PEG-NHS was calculated for OPSS-PEG-Anti-MG. Therefore, it contains the shift induced by anti-MG and the polymer. There is no trend relating the molecular weight and the amount of anti-MG binding to the polymer. This can be explained by the fact that the layer preparation can induce some aggregation for alginic acid, humic acid, polyacrylic acid, and polylactic acid. Usually, the number of binding sites for the anti-MG increases using larger polymers as previously demonstrated by Masson et al. (ibid). An increase in sensitivity using larger CM-Dextran up to 500,000 kDa was shown. The molecular weights for polymers used in this study ranged from 2000 Da to larger than 1,000,000 Da, but the shift did not correspond to the molecular weight. For example, alginic acid had the same shift as CM-Dextran, which had a 10-fold greater molecular weight. Attempts to measure the molecular weight for PMAVA by mass spectrometry and gel-permeation chromatography were not successful. Every polymer showed a shift for the anti-MG binding. The shift for polylactic acid is believed to come from nonspecific binding of anti-MG. Specifically, anti-MG may be trapped in the polymer.

The so-prepared sensors' performances in 25 ng/mL MG saline solution were measured for every polymer. FIG. 7 is an example sensorgram of antigen binding using CM-dextran. It also shows how the shift was calculated for the MG binding. A larger shift with MG denotes a more sensitive sensor. The polymers with the larger shift were CM-Dextran and alginic acid. However, every polymer showed a detectable signal for this MG solution. Only hyaluronic acid had a very weak signal of 0.02 nm, which is the detection limit with the system used. The signal for polylactic acid came from nonspecific binding of anti-MG.

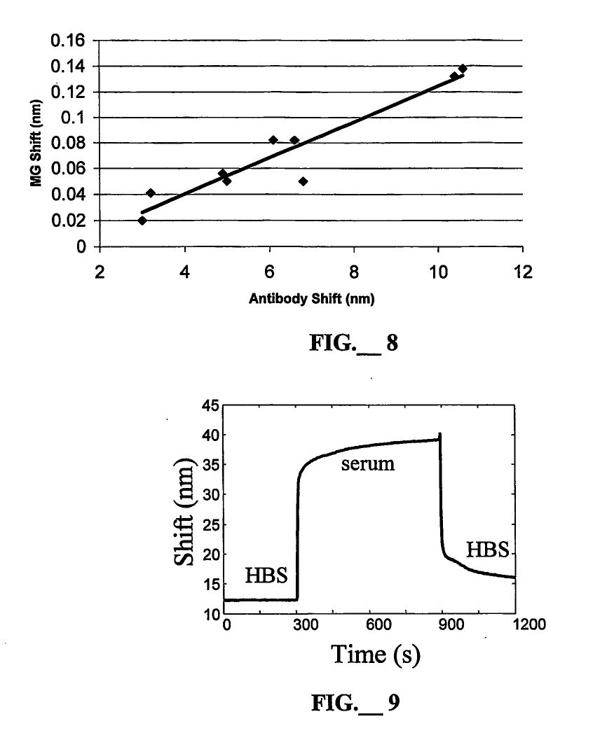

Table 2 summarizes the results obtained for anti-MG and MG performance. There was an interesting and predictable correlation between the anti-MG shift and the MG shift. The MG shift was directly proportional to the anti-MG shift (FIG. 8). This demonstrates that the antibodies reacted similarly regardless of the underlying polymer. Every polymer used was able to detect a biologically relevant level of MG. The polymers had different molecular weights, eliminating the need for very large polymers to achieve the desired detection levels. This means that CM-dextran can be replaced, which will have a great deal of interest for large scale manufacturing of the sensors. It will eliminate the dextran solution that is very viscous and hard to manipulate. Many different polymers can be used for biosensors in general. Conversely, use of the polymers is not limited to SPR, the polymers can also be used in electrochemistry, localized surface plasmon resonance (LSPR) or evanescent field fiber-optic fluorescence. It also shows that changing the specific polymers does not interfere with the performance of the antibodies.

TABLE 2
Sensor performance for anti-MG binding and MG
detection with different biocompatible polymers

| | Molecular weight | Antibody Shift | |
|---|---|---|---|
| Polymer | (Da) | (nm) | MG Shift (nm) |
| OPSS-PEG-NHS | 2000 | 6.6* | 0.082 |
| Polyacrylic acid | 50,000 | 6.8 | 0.050 |
| CM-Dextran | 500,000 | 10.4 | 0.132 |
| CM-Hyaluronic | >1,000,000 | 6.1 | 0.082 |
| acid | | | |
| Hyaluronic acid | >1,000,000 | 3.0 | 0.020 |
| Alginic acid | 12,000-80,000 | 10.6 | 0.138 |
| PMAVA | N/A | 5.0 | 0.050 |
| Humic acid | 2,000-500,000 | 3.2 | 0.041 |
| Polylactic acid | 330,000-600,000 | 4.9 | 0.056 |

*Includes the shift from the polymer

### Example 9

Reaction with a Complex Solution

To use SPR sensors in complex solutions, such as serum or blood, the signal from serum or blood must be negligible. As shown in FIG. 9, CM-dextran sensors foul quickly in a complex solution. For this plot, a sensor was placed in a bovine serum solution for 10 minutes and the output was monitored. The signal for the bovine serum was around 10-100 times the signal of cTnI or MG at the low ng/mL concentration range. Therefore, the signal from the antigen cannot be detected in a serum solution. Bovine serum was used for its low cost and for its protein concentration similarity to that found in human serum.

As a further test of the possibility of distinguishing the antigen signal from the signal due to nonspecific binding of serum proteins, a dual sensor system was assembled with a reference sensor to account for serum fouling. One of the sensors had antibodies on its surface (sensing) and the other had CM-dextran alone (reference). However, the signal from a serum solution spiked with the antigen was so large that simple probe-to-probe variance was large enough to “mask” the signal from antigen binding. A reference probe did not eliminate the background signal from serum. The sensor was also placed in contact with HBS for 14 days in the same conditions as with serum. No difference in the signal was noted with HBS after 14 days exposure.

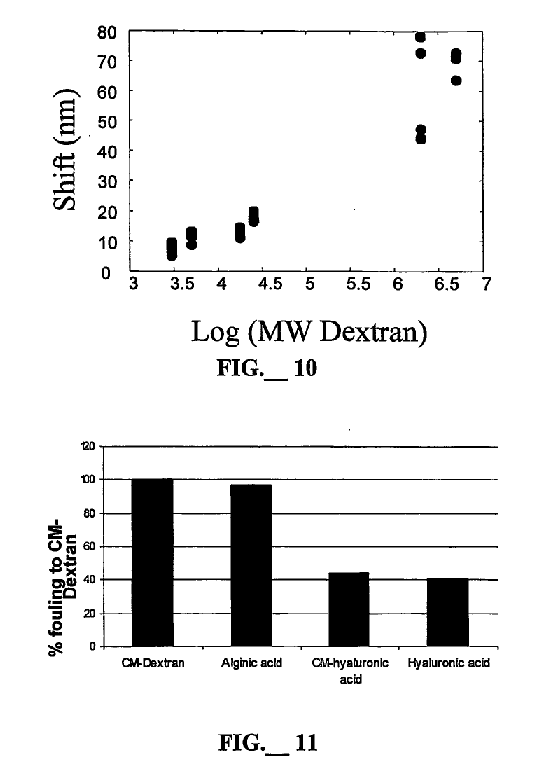

A set of sensors without immobilized antibodies was prepared to compare with sensors lacking the immobilized antibodies. The signal was statistically the same for the sensors with or without antibodies. This ruled out the possibility of localized fouling on the antibodies. FIG. 10 shows that the signal from serum is the same for both anti-MG functionalized sensors and CM-dextran only sensors. Therefore, using this method to investigate the fouling of the polymer gives a correlation independent of the amount of antibodies bounded to the surface. When sensors foul in serum, there is believed to be an electrostatic attraction between the proteins and the negatively charged polymer. The polymer molecular weight influences the fouling, such that larger polymers will show more fouling because they can physically trap more serum proteins than smaller polymers (steric interactions). Non-specific binding to the antibodies was also possible, but this was a minor fouling effect compared to the interactions with the polymer. This work demonstrated that changing the polymeric support can have a significant effect on probe fouling by proteins in solution.

The polysaccharide sensors were prepared as described above. Also as described above, anti-MG functionalized sensors were placed in a bovine serum solution at 0° C., and measurement of SPR was made daily for 14 days. Every sensor was measured once a day. The time required to measure the signal for each sensor was about 30 seconds. Measuring every sensor took about 10 minutes; therefore, the measurements were considered simultaneous. The sensor to sensor variability was 0.5 nm. The serum in this experiment came from a single batch.

As shown in FIG. 11, CM-dextran showed the worst fouling performance. Alginic acid produced results very similar to those produced when CM-dextran was used. The fouling of each candidate polymer was normalized to that observed when CM-Dextran 500 was applied, and the amount of fouling decreased as follows from CM-Dextran 500 (100%)>Alginic acid (97%)>CM-Hyaluronic acid (44%)>Hyaluronic acid (41%). This demonstrated clearly that a sensor's fouling in serum can be greatly reduced. CM-hyaluronic acid demonstrated 41% of the fouling of CM-dextran, and was 62% as sensitive as the CM-dextran. As a result, the overall performance of the sensor was improved using CM-hyaluronic acid in place of CM-Dextran. CM-hyaluronic acid has fewer carboxylic acids on the sugar structure than CM-dextran, which explains the reduced sensitivity toward MG (fewer antibodies) but also explains the better performance in serum (reduced fouling). CM-dextran has 6 carboxylic acids per 2 sugar subunits, while CM-hyaluronic acid has 5 carboxylic acids per 2 subunits; and hyaluronic acid only has one carboxylic acid per 2 subunits. However, since the signal due to fouling from serum proteins should be as low as possible, more experimentation is needed to optimize the polymers for minimal fouling and optimal sensitivity.

To summarize, a variety of polymers were evaluated as replacements for CM-dextran as polymeric supports for biosensors. CM-hyaluronic acid, hyaluronic acid, alginic acid, humic acid, polylactic acid, polyacrylic acid, OPSS-PEG-NHS, and PMAVA were synthesized and were chemically attached to the SPR sensors. The SPR signal from the sensors was monitored to ensure that the polymers were attached to the surface. Glass slides coated with Au were treated in the same fashion as the SPR sensors. GATR-FTIR was performed on the slides to confirm the polymer attachment to the gold surface of the SPR sensors. Antibodies for MG were chemically bonded to the polymers and the sensors were immersed in 25 ng/mL MG saline solution. The best performance to detect MG were obtained for alginic acid and CM-dextran. Every polymer was able to bind anti-MG and detect biologically relevant levels of MG. Probes fabricated using CM-dextran to bind anti-MG to the sensors were unable to detect MG in serum. A series of polysaccharides were used in place of CM-dextran, and the responses of the resulting probes were monitored in serum. These showed less fouling than probes fabricated using CM-dextran. This indicates that changing the polymer supporting the antibodies on the SPR sensor can improve the sensor's performance in serum. CM-hyaluronic acid and hyaluronic acid decreased by about 60% the amount of non-specific binding on the SPR sensor. To minimize the serum fouling, the polymer must reduce the electrostatic interactions and the steric interaction between the polymer and the serum proteins, as has been shown here.

### Example 10

Antibody attachment to thiols is well known and can be performed using any of several approaches. The most popular technique uses the EDC/NHS chemistry when the linker is already on the surface. However, two different approaches can be used. The antibody can be reacted with the linker prior to the linker attachment to the sensor (Chun et al. J Chem Phys 2003, 118(7):3252-3257) or a linker with acidic groups can be pre-reacted with NHS using the Lynn method (Lynn, M. IMMOBILIZED ENZYMES, ANTIGENS, ANTIBODIES AND PEPTIDES: PREPARATION AND CHARACTERIZATION, Marcel Dekker, New York City, 1975; Ch. 1, pp 1-48). To obtain very low detection limits with the thiols, the use of a cold antibody immobilization reaction was necessary. These approaches were investigated using 16-mercaptohexadecanoic acid (MHA) mixed layers with 1-hexadecanethiol (HT). The preparation of SPR sensors with polymers is described above. The SAM monolayer preparation is described here. The bare gold surface on the SPR probe was contacted overnight with mixtures of 0.005 MHA in an 80:20 solution of ethanol and water mixed with 0.005 M HT in ethanol to form a SAM. Mixtures ranged from 0.01 MHA mole fraction to 1.00 MHA mole fraction. The bare gold surface on the SPR probe was contacted overnight with mixtures of 0.005 M NHS-MHA in ethanol and 0.005 M HT in ethanol to form a SAM. Mixtures ranged from 0.01 NHS-MHA mole fraction to 1.00 NHS-MHA mole fraction. The NHS-MHA synthesis was performed according to Lynn (ibid).

Onto the SAM SPR probes, antibodies were attached, either anti-MG or anti-cTnI, on the sensor's surface. Prior to anti-MG attachment, the surface was activated by immersion in 1:1 aqueous solutions of 0.4 M EDC and 0.01 M NHS for 10 min. An amine coupling was performed on this activated surface by reaction with a 700 μg/nl solution of human anti-MG (ICN Biomedicals, polyclonal rabbit antiserum to human MG; K_{A} and k_{A} not available). The surface was reacted in a pH 4 (10 mM sodium acetate buffer) and 37° C. antibody solution for 20 mm, in a process termed the “hot” reaction.

In a “cold” reaction, the antibody for cTnI detection was attached. The activated surface was reacted in HBS at pH 7.4 and 4° C. overnight. HBS was composed of 150 mM NaCi, 10 mM HEPES, 3.4 mM EDTA, and 0.005% Tween 20 surfactant in 18 mQ deionized water. The pH of the HBS was adjusted to 7.4 using NaOH 2M solution. The non-specifically bound antibodies were washed away, and the non-reacted sites on the polymers were deactivated by rinsing the probe with an aqueous solution of 1 M ethanolamine (pH 8.5) for 10 min.

A stock solution of cTnI (Spectral Diagnostics) was prepared in HBS (pH 7.4). The cTnI was received at 1.22 mg/mL in 20 mM tris-HCl, 500 mM NaCl, 10 mM μ-mercaptoethanol at pH 7.5. This solution was stored at −20° C. for extended periods of time. A stock solution was prepared from the preceding solution at 4.88 ng/mL in HBS pH 7.4. The cTnI solution was diluted to the desired concentration with HBS pH 7.4 and thermally equilibrated in a water bath at 25° C. for 30 minutes before analysis. The sensor was equilibrated for 15 min in HBS before use. The SPR signal was monitored for 5 min in a static HBS pH 7.4 solution for 5 min and then transferred to the analyte solution. The analyte measurement was performed in a static solution. The sensor was exposed to HBS after analyte measurement for regeneration. Up to five consecutive measurements were obtained for each sensor before antibody degradation reduced the probe sensitivity. The data acquisition was performed at a rate of one data point every three sec. Each graphed point was the sum of three data points.

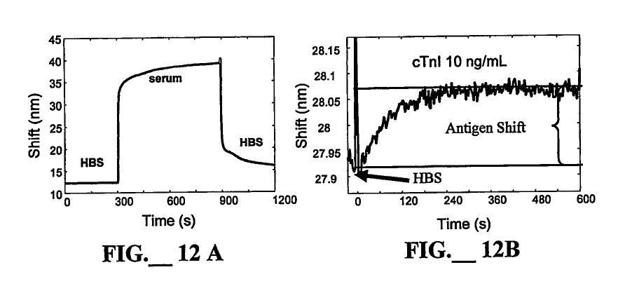

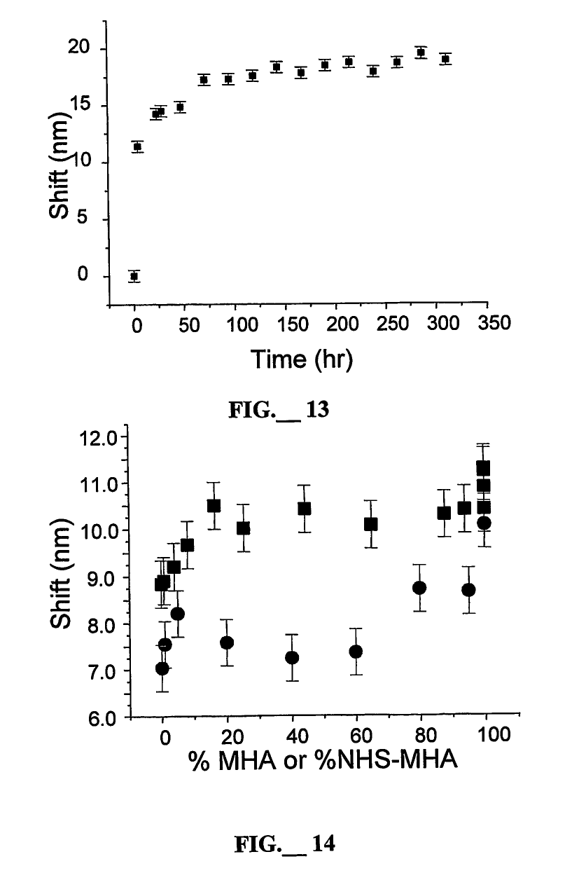

FIGS. 12A and B show the results when the cTnI sensor was placed in contact with 25° C. bovine serum containing 84 mg/mL protein (FIG. 12A) and in contact with cTnI at 10 ng/mL (FIG. 12B). The signal from protein adsorption in serum did not reach equilibrium after 10 min exposure; however, the signal for cTnI binding reached equilibrium after less than 5 min. The signal from NSB in serum was partially irreversible as seen when the sensor was washed in HBS after serum exposure (FIG. 12A).

### Example 11

CM-Dextrans of Different Molecular Weights

The molecular weight of the CM-dextran affects the binding of antigen to antibodies immobilized on the CM-dextran-coated probes, as has been studied previously. To study this effect, antibodies to cTnI were immobilized on probes that contained CM-dextran with different molecular weights. As shown in the second column of Table 3, the sensor's response to 25 ng/mL cTnI in HBS at pH 7.4 increased with the molecular weight of the CM-dextran layer up to a CM-dextran molecular weight of 500 kDa. Also, a minimum molecular weight of 150 kDa was required to detect the antigen in a 25 ng/mL cTnI solution. CM-dextran 500 klDa was used as a standard because of its commercial availability through the Biacore system.

TABLE 3
CM-dextran layer performance for cTnI detection and NSB in bovine serum

| | | | % NSB | | |
|---|---|---|---|---|---|
| | | | compared to | NSB surface | Performance |
| CM-dextran | 25 ng/mL cTnI | NSB Shift | CM-dextran | coverage | factor |
| MW (kDa) | shift (nm) | (nm) | 500 kDa | (ng/cm²) | ×10⁻³ |
| 3 | N/A | 7 ± 2 | 27 ± 9 | 2.4 | N/A |
| 5 | N/A | 11 ± 2 | 42 ± 11 | 5.9 | N/A |
| 17.5 | N/A | 12 ± 2 | 46 ± 11 | 7.1 | N/A |
| 25 | 0.000 ± 0.008 | 12 ± 2 | 45 ± 11 | 7.1 | 0 |
| 75 | 0.000 ± 0.008 | 14 ± 6 | 53 ± 24 | 9.6 | 0 |
| 150 | 0.026 ± 0.008 | 15 ± 7 | 57 ± 28 | 11 | 1.7 |
| 250 | 0.087 ± 0.008 | 29 ± 6 | 110 ± 30 | 42 | 3.0 |
| 500 | 0.150 ± 0.008 | 27 ± 5 | 100 ± 26 | 36 | 5.6 |
| 2000 | N/A | 61 ± 17 | 226 ± 76 | 191 | N/A |
| 5000 | 0.068 ± 0.008 | 69 ± 5 | 258 ± 52 | 247 | 1.0 |

MW: molecular weight;
N/A: result not available

The binding kinetics for CM-dextran 500 kDa are shown in FIG. 13. An SPR sensor coated with CM-dextran was exposed to 0° C. bovine serum with 84 mg/mL protein for 14 days. The Shift is referenced to the first data point acquired when the sensor was exposed to bovine serum. The shift reported in Table 3 is an average of the shift for the final five days of the experiment.

To study the effect of CM-dextran MW on NSB, NSB with CM-dextran layers ranging from 3 kDa to 5,000 kDA were observed in serum. As mentioned above, NSB was measured as the shift in λ_{SPR} resulting from non-specific binding (reported in n), or as the shift observed for the system of interest divided by that observed when 500 kDa CM-dextran layers were present on the SPR probes times 100 (reported as % NSB of 500 kDa dextran).

As shown in the third column of Table 1, NSB varies from 7±2 nm (27±9%) with 3 kDa CM-dextran to 69±5 nm (258:±52%) with 5,000 kDa CM-dextran. Low molecular weight CM-dextran (3-75 kDa) layers did not allow the detection of low antigen concentrations, but they are included in this study for the sole purpose of evaluating their effects on NSB. The use of mid-size CM-dextran, 150 kDa, produced significant levels of NSB; greater than 15±6 nm (57±28%) NSB was obtained compared to 500 kDa. For CM-dextran larger than 500 kDa, the NSB was too great to use in serum.

Coatings must also be compared for both their performance to detect an antigen and their ability to reduce the amount of NSB on the SPR sensor. For that purpose, a performance factor (PF) is described by equation 2.

$$PF = \frac{{A}_{shift}}{{NSB}_{Shift}} \qquad ( 2 )$$

Where A_{shift} is the shift from the detection of a 25 ng/mL antigen solution using a given surface coating and NSB_{shift} is the shift recorded in the NSB experiment for the same surface coating.

Larger values of PF indicate a more desirable coating, although surface coatings with performance factors less than 1 could be useful if their surfaces were pre-treated with BSA or serum to block NSB sites on the sensor. In this study, coatings showing a PF greater than the 500 kDa CM-dextran reference were given particular attention (see below). The PFs for different CM-dextran molecular weights are shown in the sixth column of Table 3. These were measured using a 25 ng/mL cTnI solution in HBS pH 7.4. The PF increased from 0 to 5.6×10-3 for increasing molecular weights up to 500 kDa. The optimal performance was obtained with CM-dextran 500 kDa. CM-dextran 500 kDa balanced a large sensor response and an average NSB performance compared to the other CM-dextrans. With larger CM-dextran the performance decreased, due to a loss in the sensor's response to antigen binding coupled with an increase in NSB.

The surface coverage in column 5 of Table 3 is an approximation using the calculations from Jung et al. (Langmuir, 1998, 14:5636-5648) for the thickness of the NSB adsorbed layer and from de Feijter et al (Biopolymers, 1978, 17:1759-1772) for the calculations of the surface coverage. The thickness (d) of an adsorbed layer can be calculated using equation 3. NSB Shiftₘₐₓ can be calculated from equation 4.

$$\begin{matrix}d = \frac{{l}_{d}}{2} ln ( 1 - \frac{NSBShift}{{NSBShift}_{max}} ) & ( 3 ) \\ {NSBShift}_{max} = m ( {η}_{a} - {η}_{S} ) & ( 4 )\end{matrix}$$

The value of NSB Shiftmax is obtained knowing the slope (m) of the change in the SPR signal with respect to the refractive index, the refractive index of the adsorbed layer (ηₐ), and the refractive index of the solution (n5) Using the configuration presented here, the slope is 2253 nm/RIU. The refractive index for protein is usually 1.57 [Jung et al.]. The penetration depth (I_{d}) of the surface plasmon wave is approximately 230 nm for the wavelength range used in this experiment. The NSB surface coverage (I) is calculated from equation 5. δλ/δc is the SPR minimum wavelength increment with concentration of protein. It was measured at 0.46 nm*cm³/mg.

$$Γ = \frac{d * NSBShift}{( \frac{\partial λ}{\partial c} )} \qquad ( 5 )$$

The surface coverage for the CM-dextran layer ranged from 2.4 ng/cm² for CM-dextran 3 kDa to 247 ng/cm² for CM-dextran 5000 kDa. The surface coverage is proportional to the NSB Shift; therefore, larger CM-dextran polymers have larger surface coverage due to larger amounts of non-specifically bound proteins.

The effectiveness of the polysaccharide coatings at reducing NSB was compared to that observed with 500 kDa CM-dextran (% NSB reported is the NSB for a coating of interest divided by that of CM-dextran 500 kDa times 100). Studies were performed by immersing the probes in bovine serum (84 mg/mmL protein). As shown in Table 4, CM-dextran presented a greater degree of NSB than all the other polysaccharide polymers surveyed. The performance of alginic acid was close to that of CM-dextran 500 kDa. The NSB decreased as follows: from CM-dextran 20±4 mn (100±28%)=alginic acid 20±2 nm (97±22%)>CM-hyaluronic acid 9±2 nm (44±13%)=hyaluronic acid 8 nm (41%, n=1). The influence of the surface coatings on the probe sensitivity to antigens was measured based on the probe response when immersed in 25 ng/mL MG. For these studies, anti-MG was immobilized on the probes as described above. CM-hyaluronic acid reduces NSB by 56% compared to 500 kDa CM-dextran, but is only 62% as sensitive in detecting MG compared to 500 kDa CM-dextran supports. CM-hyaluronic performance factor (PF=9.1×10-3) was only slightly greater than that of CM-dextran (PF=6.6 x 10-3). Considering NSB reduction and antigen sensitivity, the overall performance of the sensor was improved using CM-hyaluronic acid compared to 500 kDa CM-dextran. CM-hyaluronic acid has fewer carboxylic acids on the sugar structure than CM-dextran which explains the lower sensitivity towards MG, but also explains the reduced NSB in serum. Alginic acid has a PF similar to CM-dextran, 6.9×10⁻³ compared to 6.6×10⁻³ for CM-dextran. The only coating with a worse performance than CM-dextran was hyaluronic acid (PF=2.5×10⁻³).

TABLE 4
Polysaccharides layer performance for MG detection and NSB in bovine serum 84 mg/mL

| | | | | % NSB | NSB | |
|---|---|---|---|---|---|---|
| | | | | compared | surface | Performance |
| | MW | 25 ng/mL MG | NSB Shift | to CM- | coverage | factor |
| Layer | (kDa) | shift (nm) | (nm) | dextran 500 kDa | (ng/cm²) | ×10⁻³ |
| CM-dextran | 500 | 0.132 ± 0.008 | 20 ± 4 | 100 ± 28 | 20 | 6.6 |
| CM- | >1,000 | 0.082 ± 0.008 | 9 ± 2 | 44 ± 13 | 4.0 | 9.1 |
| Hyaluronic | | | | | | |
| acid | | | | | | |
| Hyaluronic | >1,000 | 0.020 ± 0.008 | 8 (n = 1) | 41 (n = 1) | 3.1 | 2.5 |
| acid | | | | | | |
| Alginic acid | 12-80 | 0.138 ± 0.008 | 20 ± 2 | 97 ± 22 | 20 | 6.9 |

### Example 12

Other Biocompatible Polymers

Six biocompatible polymers were investigated to reduce serum NSB. By defintion, “biocompatible” polymers do not cause damage or adversely affect biological function when introduced into the body. Therefore, in the SPR sensor case, blood coagulation is prevented on the sensor by the biocompatible polymer. As shown in Table 5, all of the biocompatible polymers studied showed reduced NSB compared to CM-dextran. As in Tables 3 and 4 above, performance (NSB %) is measured relative to that observed on CM-dextran 500kDa. PMAVA sustained only 14 nm (71%, n=1), polyacrylic acid was 11±2 nm (50±14%), polylactic acid was 8.4±1.1 nm (41±10%), humic acid was 7.9:0.7 nm (39±8%), and OPSS-PEG-NHS was 7.6 J 0.9 nm (36±8%). These polymers offer similar anti-NSB performance compared to the polysaccharides. The best biocompatible polymer, OPSS-PEG-NHS, has only a 5% improvement in NSB % compared to the best polysaccharide, hyaluronic acid. This is within the experimental error. Polylactic acid and OPSS-PEG-NHS do not have any carboxylic acids on the backbone; these also are low-NSB coatings. The effect of the polymer coatings on probe sensitivity to 25 ng/mL MG was tested using anti-MG immobilized on the polymers. The PFs for most biocompatible polymers were equal to or lower than CM-dextran 500 kDa, except for OPSS-PEG-NHS. OPSS-PEG-NHS has a PF of 11×10-3, almost double that of CM-dextran, and more than that of CM-hyaluronic acid.

TABLE 5
Biocompatible layer performance for MG detection and NSB in bovine serum (84 mg/mL
protein)

| | | | | % NSB | NSB | |
|---|---|---|---|---|---|---|
| | | | | compared | surface | Performance |
| | MW | 25 ng/mL MG | NSB Shift | to CM- | coverage | factor |
| Layer | (kDa) | shift (nm) | (nm) | dextran 500 kDa | (ng/cm²) | ×10⁻³ |
| CM-dextran | 500 | 0.132 ± 0.008 | 20 ± 4 | 100 ± 28 | 20 | 6.6 |
| PMAVA | N/A | 0.050 ± 0.008 | 14 (n = 1) | 71 (n = 1) | 9.6 | 3.6 |
| Polyacrylic | 50 | 0.050 ± 0.008 | 11 ± 2 | 50 ± 14 | 5.9 | 4.5 |
| acid | | | | | | |
| polylactic | 330-600 | 0.056 ± 0.008 | 8.4 ± 1.1 | 41 ± 10 | 3.4 | 6.7 |
| acid | | | | | | |
| humic acid | 2-500 | 0.041 ± 0.008 | 7.9 ± 0.7 | 39 ± 8 | 3.0 | 5.1 |
| OPSS-PEG- | 2 | 0.082 ± 0.008 | 7.6 ± 0.9 | 36 ± 8 | 2.8 | 11 |
| NHS | | | | | | |

### Example 13

Mixed SAM layers

Mixed SAM layers (MHA with HT or NHS-MHA with HT) were used for comparison with the biocompatible polymers. Sensors were prepared using multiple layer compositions for both MHA and NHS-MHA mixed with HT. As shown in FIG. 14, the composition of the mixed SAM layer varies in a non-linear fashion with the solution composition. In both the very low and very high MHA or NHS-MHA solution concentration regions, the layer composition changes significantly with solution concentration, while at intermediate solution concentrations, there is a roughly negligible effect of solution concentration on the SAM composition. This is controlled by the thermodynamics of the layer formation (Folkers, J. P.; et al. J Adhes Sci Techn 1992, 6(12):1397-1410). The range of constant SAM composition ranges from concentrations of about 20% MHA or NHS-MHA to about 90% MHA or NHS-MHA. Therefore, within this stable layer region, only one composition was employed to measure the NSB and the antigen performance. Also to be noted is the smaller shift for NHS-MHA than for MHA, where shift corresponds to the wavelength of minimum returned light from the probe in the presence of the adsorbed SAM layer compared to that in the absence of the adsorbed SAM layer. This is explained by the fact that NHS-MHA has the bulkier NHS end group, causing a less dense layer to be formed.

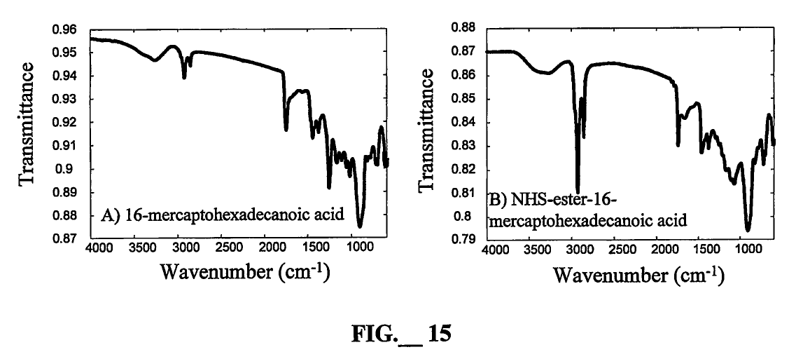

The SAM layer attachment was monitored for 100% MHA and 100% NHS-MHA using GATR-FTIR to identify the SAM species on the gold surface. A glass slide was coated with either MHA or NHS-MHA. The surface was not exposed to bovine serum. The carbonyl C═O regions were analyzed to validate the surface species. MHA displays one band at around 1745 cm⁻¹ (FIG. 15A). NHS-MHA displays 4 bands in the same region: two intense bands at 1740 cm⁻¹ and 1660 cm⁻¹, and two weak bands at 1775 cm⁻¹ and 1810 cm⁻¹ (FIG. 15B). This correlates to the FTIR of the solid compound before attachment to the gold surface and confirms the presence of the NHS group on the surface.

### Example 14

NSB properties of MHA and HT Mixed Layers

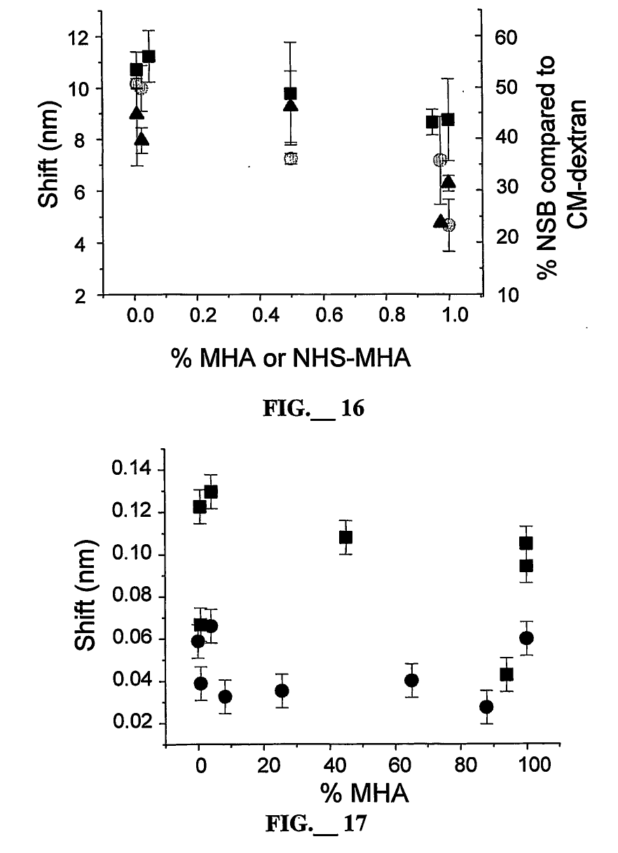

The sensors were prepared with three distinctly different SAM compositions. Two sensors were prepared at low MHA concentrations, one in the stable region where the SAM composition is independent of the solution composition and two in the high MHA concentration region. Then anti-MG antibody was added to each without intervening polymer. The hot and/or cold antibody attachment technique was used as indicated in Table 6. The results for the NSB reduction are shown in FIG. 16 and Table 6. These results summarize the average shift of three replicate sensors. As above, NSB was measured in bovine serum, and NSB % was relative to the NSB of CM-dextran 5001 kDa. Both hot and cold antibody attachment techniques described

above) were used to prepare the sensors. In FIG. 16, the difference between the hot (gray triangles) and cold (light gray circles) antibody attachment technique varied by ±5-10%. The shift in nm is shown on the left axis, and the NSB compared to CM-dextran is shown on the right axis. Less NSB was observed at higher MHA surface coverage for both the hot and cold antibody attachment. NSB was not different for NHS-MHA at low or high NHS-MHA surface coverage.

Some sensors showed a slightly better performance with the hot antibody reaction while others were better with the cold antibody technique. Overall, the two techniques produced an equivalent layer regarding serum NSB. Student's t-Test analysis comparing the two different antibody attachment performances demonstrated that the two techniques were not statistically different in NSB properties. However, the PFs for the hot antibody technique were consistently lower than the ones for the cold antibody attachment technique. This can be explained by the sensor's response difference using different antibody binding protocol (see below). Hot antibody binding may increase denaturation of the antibody (decreasing the numerator of PF). The PF values for the hot antibody technique are lower than the ones for CM-dextran with the exception of 100% MHA which more than doubled the performance compared to CM-dextran. Following the cold antibody attachment, the sensors have larger PF values than CM-dextran and ranged from 8.6×10⁻³ to 17×10⁻³. A trend can be noted, in that the sensors have better anti-NSB properties with a higher MHA concentration. A NSB reduction of about 20% compared to CM-dextran was obtained with 100% MHA compared to 1% MHA. Analysis with the Student's t-Test demonstrated the statistical difference between layers of 1% and 2.5% MHA compared to 100% MHA validating the NSB reduction. An NSB shift reduction of 16 nm (78% compared to CM-dextran) was obtained using 97.5% MHA with the cold antibody attachment and with 100% MHA using the hot antibody attachment.

TABLE 6
SAM layer performance for MG detection and NSB in bovine serum (84 mg/mL)

| | | | % NSB | | |
|---|---|---|---|---|---|
| | 25 ng/mL | NSB | compared to | NSB surface | Performance |
| | MG shift | Shift | CM-dextran | coverage | factor |
| Layer | (nm) | (nm) | 500 kDa | (ng/cm²) | ×10⁻³ |
| CM-dextran 500 kDa | 0.132 ± 0.008 | 21 ± 4 | 100 ± 27 | 22 | 6.3 |
| 1% MHA hot | 0.039 ± 0.008 | 10.2 ± 0.2 | 48 ± 9 | 5.1 | 3.8 |
| 2.5% MHA hot | 0.066 ± 0.008 | 10.0 ± 0.9 | 47 ± 10 | 4.9 | 6.6 |
| 50% MHA hot | 0.040 ± 0.008 | 7.2 ± 0.2 | 34 ± 6 | 2.6 | 5.6 |
| 97.5% MHA hot | 0.027 ± 0.008 | 7.2 ± 1.7 | 34 ± 10 | 2.5 | 3.8 |
| 100% MHA hot | 0.060 ± 0.008 | 4.7 ± 1.0 | 22 ± 6 | 1.1 | 13 |
| 1% MHA cold | 0.123 ± 0.008 | 9 ± 2 | 42 ± 12 | 3.9 | 14 |
| 2.5% MHA cold | 0.130 ± 0.008 | 8.0 ± 0.5 | 37 ± 7 | 3.1 | 16 |
| 50% MHA cold | 0.108 ± 0.008 | 9.3 ± 1.4 | 44 ± 11 | 4.2 | 12 |
| 97.5% MHA cold | 0.043 ± 0.008 | 5 (n = 1) | 22 (n = 1) | 1.1 | 8.6 |
| 100% MHA cold | 0.105 ± 0.008 | 6.3 ± 0.3 | 29 ± 6 | 1.9 | 17 |
| 1% NHS-MHA | 0.000 ± 0.008 | 10.7 ± 0.7 | 50 ± 10 | 5.6 | 0.0 |
| 5% MHA-MHA | 0.045 ± 0.008 | 11.2 ± 1.0 | 53 ± 11 | 6.2 | 4.1 |
| 50% NHS-MHA | 0.090* ± 0.008 | 10 ± 2 | 46 ± 13 | 4.7 | 9.0 |
| 95% NHS-MHA | 0.155 ± 0.008 | 8.7 ± 0.5 | 41 ± 8 | 3.7 | 18 |
| 100% NHS-MHA | 0.143 ± 0.008 | 8.7 ± 1.6 | 41 ± 11 | 3.7 | 16 |

*Signal from a 30% NHS-MHA layer. The signal for MG is constant in the region from 20% NHS-MHA to 70% NHS-MHA.

### Example 15

Anti-NSB properties of NHS-MHA and HT Mixed Layers

Mixed SAM layers (with NHS-MHA and HT) were reacted using the cold antibody method only. The pre-attachment of NHS on MHA has proven useful for the use of cold antibody attachment (Chun, K -Y. et al. J Chem Physics 2003, 118(7):3252-3257). Similarly to MHA mixed layers, two sensors were prepared in the low NHS-MHA concentration region, one in the stable region where the SAM composition is independent of the solution composition and two in the high NHS-MHA concentration region. The NSB performance of these probes is shown in FIG. 16 and Table 6. As in the work above, NSB % is measured relative to the NSB of a CM-dextran 500 kDa coating. The NSB ranged from 10.7±0.7 nm (50:10%) for 1% NHS-MHA to 8.7±1.6 nm (41:11%) for 100% NHS-MHA. NHS-MHA had a higher degree of NSB than MHA at high MHA concentration but a reduced NSB at low MHA concentration. A 9% NSB reduction was noted for 100% NHS-MHA in comparison to 1% NHS-MHA. The Student's t-Test revealed that there was no statistical difference between low and high % NHS-MHA. NHS-MHA was better than CM-dextran, but its NSB was worse than MHA.

The PFs for NHS-MHA layers range from 0 to 18×10^{-3.} The lower PFs were observed with low % NHS-MHA layers, while higher PFs were observed for 95% and 100% NHS-MHA. The NSB for every NHS-MHA layer was constant through the concentration range. Low % NHS-MHA has a low response to MG, and therefore a low PF. The highest PFs for NHS-MHA layers were a three-fold improvement compared to CM-dextran. These PFs were the highest reported in this study.

The polysaccharides and the biopolymers demonstrated worse NSB reduction properties. This means that high molecular weight and charged coatings enhanced serum protein adsorption. The lowest amount of NSB was obtained using MHA. Lower molecular weight coatings reduced the amount of NSB. Also, a reduction of NSB was observed with higher % of MHA compared to layers mainly composed of HT layer. This indicates that a more hydrophobic layer (high HT percentage) did not reduce the NSB as much as a hydrophilic layer did. MHA is therefore the best choice for surface coating because it is smaller and less charged than the polysaccharides and the biopolymer, but it is hydrophilic enough to reduce NSB.

### Example 16

Sensor Performance to Detect MG with Sensors Prepared by the “Cold” and “Hot” Antibody Reactions

With MHIHT mixed layers, cold and hot antibody fixation was compared by sensor performance in detecting biological levels of MG. A 25 ng/mL MG solution in HBS pH 7.4 was used for this experiment. Mixed layers ranging from 1% MHA to 100% MHA were prepared. FIG. 17 showed a constant signal for both the cold and hot antibody fixation reactions. The cold reaction had more spread in the results. The average shift for the hot antibody reaction across the composition range was 0.045±0.015 nm (n=8), while for the cold antibody reaction, the average shift for MG detection was 0.096±0.031 nm (n=7). More than a two-fold increase in the sensor's response was seen for MG sensing using the cold antibody attachment. A Student's t-Test was performed on the two data sets to verify the difference between the two methods. The means were considered different to 99% significance, t_{0.01; 13}=3.01 compared to tculated=4.06. The hot antibody attachment used the physiologic temperature, 37° C., and acidic pH 4 that together tend to degrade anti-MG on long exposure. The cold antibody reaction used the normal storage conditions for anti-MG, which are pH 7.4 and 4° C. Anti-MG is stable for a month in these latter conditions. There was no degradation during the anti-MG attachment during the cold antibody attachment. The only drawback is the prolonged reaction time for the cold antibody reaction. The cold antibody technique requires an overnight reaction to obtain a large antibody surface coverage, while with the hot antibody reaction takes only 20 minutes.

### Example 17

Antigen Performance

Antigen performance was measured with a 25 ng/mL MG or cTnI solution in HBS pH 7.4. CM-dextran and biocompatible polymers performance to detect an antigen have been described above. The shift is used instead of the surface coverage to compare the antibody binding to the sensor because the shift compares the relative performance between probes, and this is easily correlated using the shift during antibody-antigen binding. The surface coverage does not tell how close to the surface the coverage is. SPR sensitivity decreased when the coverage is further from the surface. Hence the SPR sensitivity may be different than the surface coverage. CM-dextran performance increased for molecular weight up to 500 kDa, the maximum shift being 0.150±0.008 nm. The sensor performance was measured by the signal from a 25 ng/mL cTnI solution in HBS pH 7.4. Biocompatible polymers had different performances for the detection of a 25 ng/mL MG solution in HBS pH 7.4, ranging from 0.020±0.008 nm for hyaluronic acid to 0.138±0.008 nm for alginic acid. In general, the performance was between 0.040±0.008 nm to 0.080±0.008 nm. These values were obtained using probes prepared by the hot antibody reaction. A concentration dependent profile for NHS-MHA can be observed that can be correlated to the surface coverage of NHS-MHA. MHA does not have a surface coverage dependent profile.

### Example 18

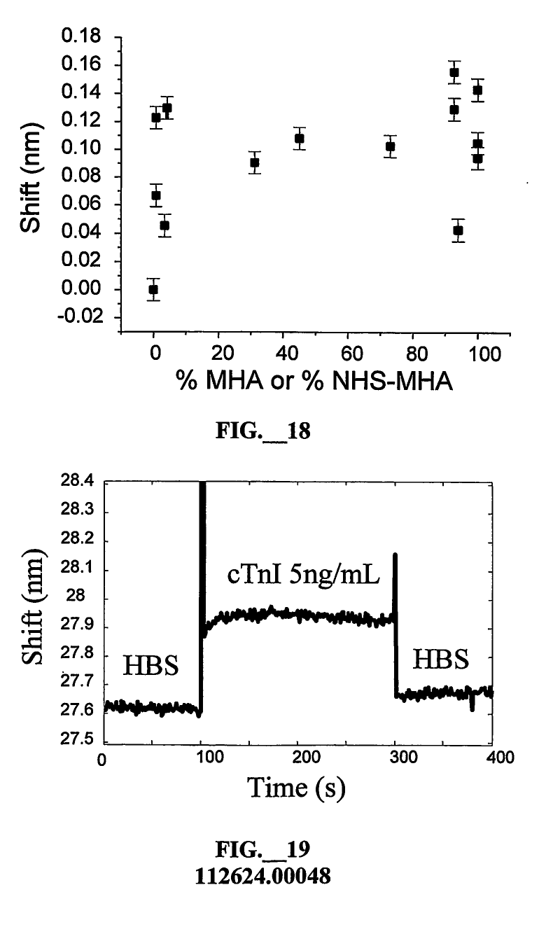

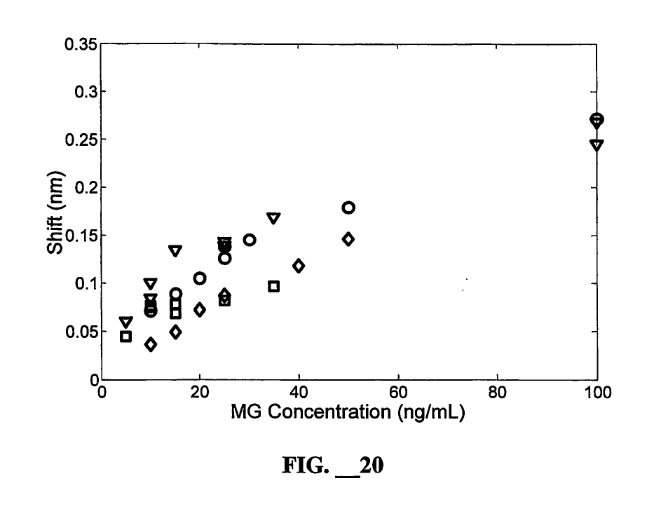

Thiols have been reputed to lack sensitivity. However, in these experiments, the use of a thiol in the cold antibody method has improved the sensor response to as good as the best polymeric supports. With MHA 0.130±0.008 nm was obtained, and with NHS-MHA 0.155±0.008 nm was obtained. The latter was 17% better than 500 kDa CM-dextran support (0.132±0.008 nm) that is well known for its great sensor response. Another interesting result is that NHS-MHA performance correlates to the NHS-MHA solution composition (FIG. 18). Using MHA, the performance was independent of the MHA percentage on the surface. Surprisingly every tested support was able to attach enough antibodies to detect MG at 25 ng/mL.

### Example 19

Sensor Calibrations

Sensor calibrations were performed using MG solutions from 5 ng/niL to 100 ng/mL in HBS pH 7.4. CM-dextran using the hot antibody attachment [32] and sensors with OPSS-PEG-NHS, 100% NHS-MHA or alginic acid using the cold antibody reaction were calibrated. The calibration was done using a Langmuir isotherm (Eq. 6).

$$\frac{1}{Shift} = \frac{1}{{Shift}_{max} KC} + \frac{1}{{Shift}_{max}} \qquad ( 6 )$$

where Shift is the change in the minimum SPR wavelength (nm), Shiftₘₐₓ is the maxinum change in the minimum SPR wavelength for a total antigen coverage on the sensor, C is the concentration of antigen in solution (ng/mL), and K is the affinity constant for the antigen-antibody system.

Every assumption in the Langrmuir model is satisfied in our model case These assumptions are that only one molecule can be adsorbed per site, only one type of site is present, the adsorption of one molecule does not affect the adsorption energy of the other molecules, only one adsorbing species is present, the solution is dilute, and the adsorption is reversible (Gonzales, N. R. et al. J Immunol Meth 2002, 268:197-210). The reversibility of the adsorption is demonstrated in FIG. 19. The sensors showed different sensitivities to MG. When 1/Shift was plotted as a function of 1/Shiftₘₐₓ, the slope of the resulting line provides an estimate of the sensitivity for a given solution concentration of antigen. The greatest sensitivity in the biologically relevant range, 15-30 ng/mL, was for alginic acid followed by CM-dextran 500, 100% NHS-MHA, and OPSS-PEG-NHS (FIG. 20 and Table 7). The detection time (maximum binding at equilibrium) is slightly longer for all tested coatings compared to CM-dextran. CM-dextran required about 5 min to equilibrate while the other coatings required about 10 min. However, these times are a small fraction of the delay with current test methods. Every coating showed enough sensitivity to detect biologically relevant concentrations of MG. During M's, MG reaches levels to approximately 15 ng/mL to 30 ng/mL or higher for serious MI damage.

TABLE 7
Coating calibration using MG in HBS pH 7.4

| | Sensitivity | 1/Shiftₘₐₓ | Concentration range |
|---|---|---|---|
| Coatings | (ng/mL)⁻¹ nm⁻¹ | nm⁻¹ | ng/mL |
| Alginic acid | 260 | 1.6 | 10-50 |
| CM-Dextran | 113 | 3.1 | 10-100 |
| 100% NHS-MHA | 65 | 3.8 | 5-100 |
| OPSS-PEG-NHS | 62 | 8.7 | 5-35 |

In sumunary, serum NSB on SPR probes due to the presence of different immobilized species on the probes was reduced by up to 78% using immobilized species other than CM-dextran 500 kDa. Immobilized fihns from solutions containing high MHA concentrations were responsible for the 78% reduction. The use of smaller CM-dextran greatly decreased NSB compared to CM-dextran 500 kDa; only 27±9% NSB for CM-dextran 3 kDa compared to 500 kDa. However, probes using CM-dextran 3 kDa as an antibody support/anti-NSB layer were unable to detect low concentrations of cTnI. Other polysaccharides decreased the amount of NSB to 41% of that obtained using CM-dextran 500 kDa, and they allowed MG detection at 25 ng/mL. The use of OPSS-PEG-NHS lowered the NSB adsorption (36±8% of the NSB observed for CM-dextran 500 kDa). Every tested biocompatible polymer allowed MG detection at 25 ng/mL. Interesting results were obtained with the thiol layers: MHA-NHS reduced NSB to 41±11% compared to CM-dextran 500 kDa, while MHA reduced NSB to 22±6%. Those results were obtained using a layer immobilization step with a high MHA or MHA-NHS concentration with both the cold and hot antibody binding techniques. The hot antibody binding technique generally produced less sensitive sensors than the cold technique. With the cold antibody attachment, greater than two-fold sensor response improvement was observed over the hot method. NHS-MHA sensitivity performance with the cold antibody attachment was better than that of any other coatings. It was 20% better than CM-dextran that is known for its high sensitivity. A performance factor is described to compare the surface coatings. A large performance factor exhibits a good sensor's response to detect a 25 ng/mL antigen solution and to reduce the amount of NSB. The highest performance factors were obtained using high percentages of MHA or NHS-MHA SAM layers. Calibration curves were obtained using MG and different coatings to show their performance in IBS pH 7.4. Every coating was able to detect MG at biologically relevant concentrations.

Various embodiments of the invention are described above in the Drawings and Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

1. A method of coating an SPR biosensor specific for an analyte to reduce protein fouling, the method comprising
a. providing an SPR biosensor;
b. providing a solution of 11-mercaptoundecanol;
c. incubating the SPR biosensor in the I1-mercaptoundecanol solution to form a self-assembling monolayer (SAM);
d. incubating the SPR with SAM in a solution of epichlorohydrin and diglyme;
e. incubating the SPR from step d in ethanolamine;
f. preparing a solution of EDC/NHS and a biocompatible polymer;
g. incubating the SPR of step e in the solution of step f;
h. providing a ligand specific for the analyte in a solution;
i. incubating the SPR of step g in the solution of step h to permit the ligand to react with the SPR of step g; and
j. washing the SPR of step i to remove an unreacted ligand, thereby providing an SPR capable of reacting with the analyte.

2. The method of claim 1 wherein the biocompatible polymer is prepared from carboxymethylated hyaluronic acid, OPSS-PEG-NHS, alginic acid, humic acid, polymethacrylate co-vinyl acetate or polyacrylic co-vinyl acetate.

3. The method of claim 1 wherein the analyte is an antigen and the ligand is an antibody.

4. The method of claim 3 wherein the antigen is cardiac myoglobin and the antibody is anti-myoglobin.

5. The method of claim 3 wherein the antigen is cardiac troponin I and the antibody is anti-cardiac troponin I.

6. The method of claim 3 wherein the antigen is interleukin-6 (IL-6) and the antibody is anti-IL-6, whereby the biosensor can monitor wound healing.

7. The method of claim 3 wherein the antigen is NSE and the antibody is anti-NSE, whereby the biosensor can monitor patients for ischemic stroke.

8. The method of claim 3 wherein the antigen is S-100B and the antibody is anti-S-100B, whereby the biosensor can monitor patients for ischemic stroke.

9. The method of claim 3 wherein the antigen is SMN1-4 and the antibody is anti-SMN1-4, and further comprising step k comprising preparing a cellular extract, whereby a low value is indicative of spinal motor atrophy.

10. A method of coating an SPR biosensor specific for an analyte to reduced protein fouling, the method comprising
a. providing an SPR biosensor;
b. providing a solution of MHA or NHS-MHA with HT;
c. incubating the SPR biosensor in the MHA-HT solution for a time sufficient to permit the formation of SAM;
d. providing a solution of a ligand specific for the analyte;
e. incubating the SPR biosensor with SAM in the ligand solution for a time sufficient for the ligand to react with the SAM, thereby providing the biosensor with ligands specific for the analyte.

11. The method of claim 10 wherein the analyte is an antigen and the ligand is an antibody.

12. The method of claim 11 wherein the antigen is cardiac myoglobin and the antibody is anti-myoglobin.

13. The method of claim 11 wherein the antigen is cardiac troponin I and the antibody is anti-cardiac troponin I.

14. The method of claim 11 wherein the antigen is interleukin-6 (IL-6) and the antibody is anti-IL-6, whereby the biosensor can monitor wound healing.

15. The method of claim 11 wherein the antigen is NSE and the antibody is anti-NSE, whereby the biosensor can monitor patients for ischemic stroke.

16. The method of claim 11 wherein the antigen is S-100B and the antibody is anti-S-100B, whereby the biosensor can monitor patients for ischemic stroke.

17. The method of claim 11 wherein the antigen is SMN1-4 and the antibody is anti-SMN1-4, and further comprising step k comprising preparing a cellular extract, whereby a low value is indicative of spinal motor atrophy.
